(19) United States
(12) Patent Application Publication
(10) Pub. No.: US 2022/0110903 A1
(43) Pub. Date: Apr. 14, 2022

(54) COMPOSITIONS AND METHODS FOR PREVENTING, ALLEVIATING, AND TREATING NEUROLOGICAL INJURY FOLLOWING HEAD TRAUMA

(71) Applicant: Struct Nutrition, LLC, Missoula, MT (US)

(72) Inventors: Martin Purpura, Austin, TX (US); Ralf Jäger, Whitefish Bay, WI (US); Mark Olson, Carlsbad, CA (US)

(21) Appl. No.: 17/479,743

(22) Filed: Sep. 20, 2021

Related U.S. Application Data
(62) Division of application No. 16/113,702, filed on Aug. 27, 2018.
(60) Provisional application No. 62/559,978, filed on Sep. 18, 2017.

(51) Int. Cl. A61K 31/202; A61K 9/10; A23L 33/12; A61P 25/00; A61K 9/107

(52) U.S. Cl. CPC A61K 31/202; A61K 9/10; A23V 2002/00; A61P 25/00; A61K 9/107; A23L 33/12

(57) ABSTRACT

Compositions and methods of treatment and prevention for trauma-related brain injury are disclosed herein. In certain aspects, disclosed is a method for preventing, alleviating or treating trauma-related brain injury in a subject in need thereof comprising administering to the subject a composition comprising docosahexaenoic acid and eicosapentaenoic acid.

### CROSS-REFERENCE TO RELATED APPLICATION(S)

This is a divisional application of U.S. application Ser. No. 16/113,702, filed Aug. 27, 2018, and entitled “COMPOSITIONS AND METHODS FOR PREVENTING, ALLEVIATING AND TREATING SYMPTOMS OF CONCUSSIONS,” which claims priority under 35 U.S.C. § 119(e) to U.S. Provisional Application No. 62/550,430, entitled “COMPOSITIONS AND METHODS FOR PREVENTING, ALLEVIATING AND TREATING SYMPTOMS OF CONCUSSIONS,” filed Aug. 25, 2017, and U.S. Provisional Application No. 62/559,978, entitled “COMPOSITIONS AND METHODS FOR PREVENTING, ALLEVIATING AND TREATING SYMPTOMS OF CONCUSSIONS” filed Sep. 18, 2017, each of which is hereby incorporated by reference in its entirety for all purposes.

### TECHNICAL FIELD

The disclosure relates generally to compositions and methods of treatment and prevention for concussion and sub-concussive related brain injury.

### BACKGROUND

The U.S. Centers for Disease Control and Prevention estimates that about 1.6 to 3.8 million concussions occur in sports and recreational activities annually in the United States. Concussions affect a wide range of sports and athletes from professional players to little leaguers and, therefore, have become a public health concern. It also is reported by research findings that as of consequences returning to play too soon may subject the athlete to further injury increasing likelihood of long-term effects. Recognizing concussion and providing proper treatment is especially important for younger athletes, because it typically takes them longer than adults to fully recover. Concussions result from direct or indirect impact to the head, which may occur with or without loss of consciousness and can lead to temporary cognitive symptoms. Symptoms may include headache, confusion, lack of coordination, memory loss, nausea, vomiting, dizziness, ringing in the ears, sleepiness, and excessive fatigue. There's no specific cure for concussion.

The issue of concussive, as well as sub-concussive injury is particularly important in American football. Secondary to participation in American football, athletes are routinely subject to repetitive head impacts (RHI). Though estimated that between 1.1 and 1.9 million sports- and recreation-related concussions occur annually in young athletes, those statistics do not reflect those athletes participating in contact sports who are exposed to RHI but do not sustain a clinically evident concussion (i.e., those impacts that do not result in identifiable functional disturbances or symptoms). There is a growing body of evidence linking subconcussive RHI sustained by contact-sport athletes to the development of the neurodegenerative disorder, chronic traumatic encephalopathy (CTE). While exposure to RHI via contact-sport participation may indeed be an antecedent for the development of CTE, the scientific community's understanding of CTE is limited, and to date, reliance on postmortem case-series investigations prohibits a clear understanding of the pathogenesis. Nonetheless, the general public is apparently concerned about conceivable long-term consequences of participating in American football as data from the National Federation of State High School Associations annual participation survey indicates that participation in 11-player tackle football has declined each of the past three years.

The accumulation of subconcussive RHI acquired over the course of a competitive season of contact-sport participation, even in the absence of overt functional disturbances or a clinically diagnosed concussion, results in quantifiable microstructural and functional changes in the brain. Specifically, radiographically detected white matter (WM) changes are linked to RHI sustained by American football athletes at the high school and collegiate level. Rather than rely on costly advanced neuroimaging techniques, advances in ultrasensitive single-molecule array technologies make possible the detection of biomarkers in circulating blood that are linked to RHI and diffuse microstructural injury.

There is a need in the art for compositions and methods of treatment to reduce the acute and chronic impact of concussive and sub-concussive brain injuries in individuals at high risk for such injuries.

### BRIEF SUMMARY

Described herein are various embodiments relating to compositions and methods for the treatment and prevention of trauma-related brain injury. A method is disclosed for preventing, alleviating or treating a trauma-related brain injury in a subject comprising administering to the subject a composition comprising docosahexaenoic acid (DHA); and eicosapentaenoic acid (EPA). In certain aspects, the DHA and EPA are present in a ratio ranging from about 10:1 to about 1:10. In certain aspects, the composition is administered in a therapeutically effective amount. In further aspects, the composition is administered in a prophylactically effective amount.

In certain aspects, DHA and EPA are present in a ratio of about 5:1. In further aspects, the DHA and EPA are present in a ratio of about 3.5:1.

According to certain aspects, the composition further comprises docosapentaenoic acid (DPA). In certain aspects, DHA and DPA are present in a ratio of about 7:1.

In certain aspects, the composition comprises an emulsion. In certain aspects of these embodiments, the composition further comprises one or more emulsifying agents.

According to certain aspects, the composition is administered in a dose of about 2 g per day. In further aspects, the composition comprises from about 750 mg to about 6,000 mg DHA.

In certain aspects, the composition is administered to a subject at high risk for sub-concussive injury. In still further aspects, the composition is administered to a subject at high risk for concussive injury. In yet further aspects, the composition is administered prior to the high risk of concussive injury and at regular intervals following the onset of risk for concussive injury.

In still further aspects, administration of the composition to the subject results in a synergistic reduction in a concussive biomarker relative to subjects administered DHA or EPA alone. In certain aspects, the concussive biomarker is serum Neurofilament Light (NFL). In yet further aspects, administration of the composition to the subject results in a synergistic increase in plasma levels of DHA and/or EPA relative to subjects administered comparable amounts of DHA or EPA alone.

In certain aspects, the composition is administered a nutritional supplement. In further aspects, the composition is administered a functional food.

Further disclosed is a method for attenuating the impact of repeated head impacts on the brain of a subject in need thereof comprising administering to the subject a composition comprising: DHA, EPA, and DPA, wherein the ratio of DHA:EPA:DPA is about 7:2:1. In certain aspects, the method comprises administering an amount of the composition to the subject effective to decrease the ratio of plasma ω-6FA:ω-3FA of the subject.

Further disclosed herein is a composition for attenuating repeated head impact related brain injury comprising DHA, EPA, DPA, and a pharmaceutically acceptable carrier thereof, wherein the ratio of DHA:EPA:DPA is about 7:2:1. In certain aspects, the pharmaceutically acceptable carrier comprises a phospholipid carrier

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 shows the effect of certain disclosed formulations on the percent change from baseline in serum NFL in American football athletes over the course of the season, according to certain embodiments.

FIG. 2 shows the effect of a certain disclosed formulations on the percent change from baseline in serum NFL in American football athletes over the course of the season compared to a standard 2 g dose of DHA, according to certain embodiments.

FIG. 3 shows effect of certain disclosed formulations and DHA on the relative change from 1 (T1) in serum fatty acid DHA in American football athletes over the course of the season compared to the end of the season, according to certain embodiments.

FIG. 4 shows the effect of certain disclosed formulations and DHA on the relative change from 1 (T1) in serum fatty acid EPA in American football athletes over the course of the season compared to the end of the season, according to certain embodiments.

FIG. 5 shows the effect certain disclosed formulations and DHA on the relative change from 1 (T1) in serum Arachidonic acid (ARA) in American football athletes over the course of the season compared to the end of the season, according to certain embodiments.

FIG. 6 shows the effect of certain disclosed formulations on the proportion EPA, DHA, and omega-6:omega-3 ratio, according to certain embodiments.

FIG. 7 shows the effect of certain disclosed formulations on serum NFL, according to certain embodiments.

FIG. 8 shows the effect of certain disclosed formulations on serum NFL in those American football athletes categorized as starters, according to certain embodiments.

FIG. 9 shows a schematic of an experimental design, according to certain embodiments.

FIG. 10 shows protein network interaction data, according to certain embodiments.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Docosahexaenoic acid (DHA) means the omega-3 fatty acid having the formula: C₂₂H₃₂O₂. Chemically, DHA is a carboxylic acid with a 22-carbon chain and six cis double bonds with the first double bond is located at the third carbon from the omega end. DHA can be sourced or manufactured from fats and oils of marine animals, fish oils (such as mackerel oil, menhaden oil, salmon oil, capelin oil, tuna oil, sardine oil, hill oil or cod oil), marine algae such as Schizochytrium sp., human milk, and vegetable oils, such as linseed oil, either as itself or in the form of a derivative such as a triglyceride.

Eicosapentaenoic acid (EPA) means the omega-3 fatty acid having the formula C20H30O2. Chemically, EPA is a carboxylic acid with a 20-carbon chain and five cis double bonds; the first double bond is located at the third carbon from the omega end. Because of the presence of double bonds, EPA is a polyunsaturated fatty acid. Sources of EPA include, but are not limited to, fish oils of cod liver, krill, herring, mackerel, salmon, menhaden and sardine.

Arachidonic acid (ARA) means the omega-6 fatty acid having the formula C₂₀H₃₂O₂. Chemically, ARA is a carboxylic acid with a 20-carbon chain and four cis double bonds; the first double bond is located at the sixth carbon from the omega end. ARA can be found in poultry, eggs, fish, and beef.

As used herein, docosapentaenoic acid (n-3 DPA) means the omega-3 fatty acid intermediary between EPA and DHA having the formula C22H34O2. Chemically, n-3 DPA is a precursor to DHA and is a polyunsaturated fatty acid with a 22-carbon chain and five cis double bonds. Sources of n-3 DPA include, but are not limited to, menhaden oil, salmon oil, krill oil, salmon, beef, and human breast milk.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term, “trauma-related brain injury” means an injury to the brain resulting from an external mechanical force or forces. Such force(s) may including, but are not limited to, rapid acceleration or deceleration, impact, blast waves, or penetration by a projectile. Injuries may include both those caused directly by the force on the brain or impact of the brain against the skull (e.g. coup and contrecoup injuries) resulting from the force. The term “trauma-related brain injuries” encompass those injuries referred to in the art as traumatic brain injury, concussive brain injuries, as well as sub-concussive brain injuries (e.g. trauma induced brain injuries that do not present with symptoms that meet the threshold for diagnosis of concussion or traumatic brain injury). Activities associated with high incidents of and/or high risk of trauma-related brain injury include, but are not limited to: cycling; football; baseball and softball; basketball; water sports (e.g., diving, scuba diving, surfing, swimming, water polo, water skiing, water tubing); use of powered recreational vehicles (ATVs, dune buggies, go-carts, mini bikes, off-road); soccer; skateboards/scooters; fitness/exercise/health club; winter sports (skiing, sledding, snowboarding, snowmobiling); horseback riding; gymnastics/dance/cheerleading; golf; hockey; trampolines; rugby/lacrosse; roller and inline skating; and ice skating.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “pharmaceutically acceptable carrier” or “carrier” refers to sterile aqueous or nonaqueous solutions, colloids, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil), phospholipids or mixtures of phospholipids, and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Disclosed herein is a method of preventing, alleviating or treating a trauma-related brain injury of a subject in need thereof comprising administering to the subject a composition comprising DHA and EPA (DHA/EPA). In certain aspects, the composition is administered in an effective amount. In further aspects, the composition is administered in a therapeutically effective amount. In still further aspects, the composition is administered in a prophylactically effective amount.

According to certain embodiments, DHA and EPA are present in a ratio ranging from between about 10:1 to about 1:10. According to further embodiments, DHA and EPA are present in a ratio of about 5:1 to about 1:1. In further embodiments, DHA and EPA are present in a ratio of about 3.5:1. In still further embodiments, the ratio of DHA to EPA is about 1:2.2 to about 1:1.5.

According to certain embodiments, the composition further comprises docosapentaenoic acid (DPA). In certain aspects of these embodiments, the DPA is present in a DHA:DPA ratio of about 7:1. In further aspects of these embodiments, the DPA is present in a DHA:EPA:DPA ratio of about 7:2:1.

According to certain further embodiments, disclosed is a composition for attenuating RHI related brain injury comprising docosahexaenoic acid (DHA); eicosapentaenoic acid (EPA); and docosapentaenoic acid (DPA). In certain aspects, the ratio of DHA:EPA:DPA is about 7:2:1. In further aspects, the composition further comprises a pharmaceutically acceptable carrier thereof.

According to certain further embodiments, composition is an emulsion. In certain aspects, the emulsion is prepared using one or more emulsifiers known in the art. According to exemplary embodiments, the composition contains: xylitol glycerin, gum arabic, guar gum, and/or xanthan gum. According to further embodiments, the disclosed composition can be prepared as an emulsion with the emulsifiers and processes described in McClements and Jafari, Advances in Colloid and Interface Science 251 (2018) 55-79, which is incorporated herein by reference for all purposes.

In some embodiments the carrier comprises a phospholipid or mixture of phospholipids. If certain embodiments, the phospholipid may further comprise an acceptable solubilizing agent for the phospholipid. It will be understood that reference in the singular to a (or the) phospholipid, solubilizing agent or other formulation ingredient herein includes the plural; thus combinations, for example mixtures, of more than one phospholipid, or more than one solubilizing agent, are expressly contemplated herein. The solubilizing agent, or the combination of solubilizing agent and phospholipid, also solubilizes the DHA:EPA:DPA, although other carrier ingredients, such as a surfactant or an alcohol such as ethanol, optionally present in the carrier can in some circumstances provide enhanced solubilization of the composition. In certain aspects, any pharmaceutically acceptable phospholipid or mixture of phospholipids can be used. In general such phospholipids are phosphoric acid esters that yield on hydrolysis phosphoric acid, fatty acid(s), an alcohol and a nitrogenous base.

In certain aspects, the disclosed composition contains one or more antioxidants. In certain embodiments, the disclosed composition contains the antioxidants ascorbyl palmitate and/or beta carotene. As will be appreciated by those skilled in the art, other suitable antioxidants may be employed. In still further embodiments, the composition includes one or more stabilizers (e.g. potassium sorbate). As will be appreciated by those skilled in the art, other suitable stabilizers may be used.

In certain aspects, the composition is administered at a dose range of between about 500 milligrams (mg) per day to about 6 g per day. In further aspects, the composition is administered in a dose of about 2 g per day. According to still further aspects, the composition administered to the subject comprises from between about 750 mg to 6,000 mg DHA.

According to certain exemplary embodiments of these aspects, the composition is administered between 1 and 3 times in a day.

In certain aspects of the instantly disclosed method, the composition is administered to a subject at high risk for concussive injury. In further aspects, the composition is administered to a subject at high risk for sub-concussive injury. In further aspects, the composition is administered prior to the high risk of concussive injury or sub-concussive injury. In exemplary aspects of these embodiments, the composition is administered at regular intervals following the onset of risk for concussive injury.

According to further embodiments, the instantly disclosed compositions and methods are employed to treat injuries associated with head trauma. Exemplary injuries treated by the disclosed methods include, but are not limited to, mild traumatic brain injury (TBI), moderate TBI, and severe TBI.

According to certain embodiments, administration of the compositions disclosed herein to a subject in need thereof result in a synergistic reduction in a concussive biomarker relative to subjects administered DHA or EPA alone. In certain exemplary embodiments, the concussive biomarker is serum Neurofilament Light (NFL). In these embodiments, NFL detected in the serum of a subject is utilized as a surrogate marker for axonal injury associated with head trauma. In exemplary embodiments, elevated levels of serum NFL following a potential head injury compared to baseline serum NFL are indicative of an injury having occurred. In these embodiments, elevated NFL levels may be viewed a indicative of a need for treatment by the instantly disclosed methods. In further embodiments, changes NFL levels overtime may be used to evaluate the efficacy of a given course of treatment and/or make adjustments to a course of treatment.

In certain aspects, administration of the composition to the subject results in a synergistic reduction in a concussive biomarker (e.g., serum NFL) relative to subjects administered comparable amounts of DHA or EPA alone.

According to certain aspects, administration of the composition to the subject results in a synergistic increase in plasma levels of DHA and/or EPA relative to subjects administered comparable amounts of DHA or EPA alone.

In another embodiment, upon treatment with an instantly disclosed composition for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits decreased serum NFL levels; a decreased ratio of plasma ω-6FA:ω-3FA; and/or decreased signs or symptoms of concussive or sub-concussive brain injury relative to control subjects or groups.

As will be appreciated by a person having skill in the art, the composition may be administered in a number of dosage forms. In certain aspects, the composition is administered a nutritional supplement. In further aspects, the composition is administered a functional food. According to still further aspects, the composition is administered as a part of a pharmaceutical preparation. A person having skill in the art will appreciate many such preparations are possible.

According to further embodiments, disclosed is a method for attenuating the impact of repeated head impacts on the brain of a subject in need thereof comprising administering to the subject a composition comprising docosahexaenoic acid (DHA); eicosapentaenoic acid (EPA); and docosapentaenoic acid (DPA). According to certain aspects of the these embodiments, the ratio of DHA:EPA:DPA is about 7:2:1. In further aspects, the composition is administered in an amount effective to decrease the subject's ratio of plasma ω-6FA:ω-3FA.

The most recent consensus statement for concussion in sport acknowledges that little is known about sports-related concussion prevention strategies but fails to address the need for prevention and/or protection from sub-concussive injuries. Thus, strategies to protect athletes from the potential long-term effects of contact-sport induced RHI must be explored. Disclosed herein are compositions and methods of their use that confer neuroprotection in the context of RHI. Without wishing to be bound to any particular theory as to the mechanism of action, the protective function of the disclosed compositions is likely attributable to a series of mutual mechanisms. For example, the disclosed compositions may allay glutamate cytotoxicity, suppress mitochondrial dysfunction and the development of oxidative stress, decrease calcium influx, and downregulate alpha-amino-3-hydroxy-5-methyl-4-isoxazolepropionic acid (AMPA) receptor subunits, each of which are pathological characteristics of neurological trauma. Furthermore, the disclosed compositions may improve outcomes secondary to their administration via the preservation of WM which is verified via fewer beta amyloid precursor-positive axons, enhanced conservation of myelin, and protection of neurofilament morphology. Indeed, supplementation with the disclosed compositions consistently confers enhanced resilience to head trauma with functional outcomes mirroring those biological indicators of injury, even following multiple injuries.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

### Comparison DHA/EPA Formulation Vs Placebo

### Methods

Subjects. All athlete volunteers were National Collegiate Athletic Association (NCAA) Division I or Division III American football athletes cleared to participate in university athletics as determined by the team physician. Athletes injured or unable to participate in regularly schedule conditioning or competitions were also excluded.

### Study Design.

A between groups parallel design study was employed to examine the effect of a novel formulation containing DHA/EPA on serum NFL over the course of a season to include pre-season camp and the competitive season. Thirty-one (n=31) American football athletes served as the treatment group, while thirty-three (n=33) American football athletes served as the control group. The treatment group received a novel formulation containing DHA/EPA derived from Tuna Fish Oil in the amount of 2 g per day after baseline blood sampling, while the control group received no supplementation over the course of the season. Prior to the start of the season, blood was sampled at specific intervals coincident with changes in intensity, hours of contact, and coincident with those times in which the number and magnitude of head impacts have been reported to change. A baseline blood sample was taken when volunteers returned from a period of no contact (Baseline; T1) prior to pre-season training camp (Baseline; T1). A second sample was collected following pre-season training camp (Post-Camp; T2). The remaining blood samples were taken throughout the competitive season on the Monday following a Saturday game (48 hours) (T3-T6). The maximum (and median) number of head impacts per season for an NCAA American football athlete has been reported to range from 15.6 to 24 (4.8 to 7.5) and 58.5 to 86.1 (12.1 to 16.3) for practices and games, respectively.

Serum NFL. Serum NFL (Simoa™ Beta Kit) concentrations were measured using digital array technology on a Single Molecule Array (Simoa™) HD-1 Analyzer, software version 1.5 (Quanterix; Lexington, Mass.). The same lot of kits was used. Statistical Analyses. A priori power analysis was conducted using G*Power version 3.1.9 to determine the minimum sample size required to find significant changes in the proportion of DHA in total plasma fatty acids with a desired level of power set at 0.80, an α-level at 0.05, and a standardized effect size calculated from a previous pilot study. It was determined that a total of 20 subjects (5 per group) were needed to ensure adequate power.

The effects of supplementation and time on variables of interest were calculated from a mixed model analysis of variance (ANOVA) in SPSS V.22 (IBM Corporation; Armonk, N.Y.). From that model, estimates and uncertainty of the large-sample effect size for the effect of treatment and time on dependent measures were derived from the model to allow a magnitude-based approach to inference. The probability (likelihood) that a contrast was at least greater than the smallest threshold, calculated as the standardized change of 0.2 times the between subject standard deviation at baseline among all treatments was qualified as follows: 0.5%, almost certainly not; 0.5-5%, very unlikely; 5-25%, unlikely; 25-75%, possible; 75-95%, likely; 95-99.5%, very likely; 99.5%, almost certain. In the case where the majority (>50%) of the confidence interval (CI) lies between the threshold for substantiveness and the probability of benefit or harm is <5.0%, the effect was qualified trivial with the appropriate likelihood qualifier.

### Results

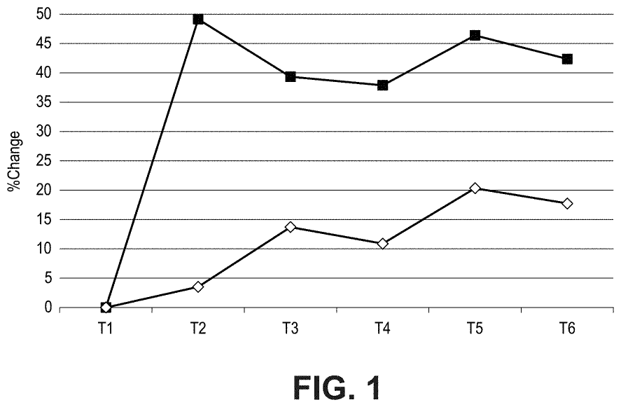

FIG. 1 shows the effect of certain disclosed formulations on the percent change from baseline in serum NFL in American football athletes over the course of the season. ▪ Placebo (n=33); 0 Supplemented athletes (n=31). Threshold for smallest substantial change calculated as 0.2 times baseline SD_{between}; Likelihood is shown as increased number of symbols and represented by unique symbol: (*) relative to placebo. * possible, ** likely, *** very likely, **** most likely. All data are means. Relative to the control group (placebo), the novel formulation very likely attenuated serum Nf-L at T2 (mean; x/÷90% confidence limits; 1.5; x/÷1.2 fold) which corresponded to post-camp, a period of significant contact. Further, the attenuation relative to placebo was likely maintained at T3 (1.3; x/÷1.2 fold), T4 (1.3; x/÷1.2 fold), T5 (1.3; x/÷1.3 fold), and T6 (1.2; x/÷1.3 fold). (FIG. 1).

### Example 2

### Comparison DHA/EPA Formulation Vs Marine Alga DHA

### Methods

Subjects. All athlete volunteers were NCAA Division I American football athletes cleared to participate in university athletics as determined by the team physician. Athletes injured or unable to participate in regularly schedule conditioning or competitions were also excluded.

### Study Design

A between groups parallel design study was employed to compare the effect of a novel formulation containing DHA/EPA to marine algae DHA on plasma serum NFL over the course of a season to include pre-season camp and the competitive season. American football athletes were assigned to ingest either a novel formulation of DHA/EPA (n=33) derived from Tuna Fish Oil or marine algae DHA (n=4) derived from Schizochytrium sp. containing 35-45% DHA by weight. Prior to the start of the season, blood was sampled at specific intervals coincident with changes in intensity, hours of contact, and coincident with those times in which the number and magnitude of head impacts have been reported to change. A baseline blood sample was taken when volunteers returned from a period of no contact (Baseline; T1) prior to pre-season training camp (Baseline; T1). A second sample was collected following pre-season training camp (Post-Camp; T2). The remaining blood samples were taken throughout the competitive season on the Monday following a Saturday game (48 hours) (T3-T6). The maximum (and median) number of head impacts per season for an NCAA American football athlete has been reported to range from 15.6 to 24 (4.8 to 7.5) and 58.5 to 86.1 (12.1 to 16.3) for practices and games, respectively.

Serum NFL. Serum NFL (Simoa™ Beta Kit) concentrations were measured using digital array technology on a Single Molecule Array (Simoa™) HD-1 Analyzer, software version 1.5 (Quanterix; Lexington, Mass.). The same lot of kits was used. Statistical Analyses. A priori power analysis was conducted using G*Power version 3.1.9 to determine the minimum sample size required to find significant changes in the proportion of DHA in total plasma fatty acids with a desired level of power set at 0.80, an α-level at 0.05, and a standardized effect size calculated from a previous pilot study. It was determined that a total of 20 subjects (5 per group) were needed to ensure adequate power.

The effects of supplementation and time on variables of interest were calculated from a mixed model analysis of variance (ANOVA) in SPSS V.22 (IBM Corporation; Armonk, N.Y.). From that model, estimates and uncertainty of the large-sample effect size for the effect of treatment and time on dependent measures were derived from the model to allow a magnitude-based approach to inference. The probability (likelihood) that a contrast was at least greater than the smallest threshold, calculated as the standardized change of 0.2 times the between subject standard deviation at baseline among all treatments was qualified as follows: 0.5%, almost certainly not; 0.5-5%, very unlikely; 5-25%, unlikely; 25-75%, possible; 75-95%, likely; 95-99.5%, very likely; 99.5%, almost certain. In the case where the majority (>50%) of the confidence interval (CI) lies between the threshold for substantiveness and the probability of benefit or harm is <5.0%, the effect was qualified trivial with the appropriate likelihood qualifier.

### Results

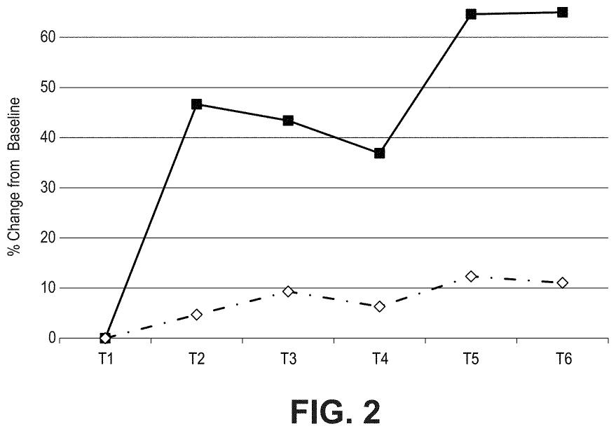

FIG. 2 shows the effect of a certain disclosed formulations (0; n=33) on the percent change from baseline in serum NFL in American football athletes over the course of the season compared to a standard 2 g dose of algal DHA (▪; n=4). Threshold for smallest substantial change calculated as 0.2 times baseline SD_{between}; Likelihood is shown as increased number of symbols and represented by unique symbol: (*) relative to standard 2 g dose of algal DHA. * possible, ** likely, *** very likely, **** most likely. All data are means. Relative to the marine algae DHA, the novel formulation likely attenuated serum NFL at T2 (mean; x/÷90% confidence limits; 1.4; x/÷1.4 fold) which corresponded to post-camp, a period of significant contact, T3 (1.3; x/÷1.4 fold), T4 (1.3; x/÷1.4 fold). (FIG. 2).

### Example 3

### Comparison DHA/EPA Formulation Vs Marine Algae DHA

### Methods

Subjects. All athlete volunteers were NCAA Division I American football athletes cleared to participate in university athletics as determined by the team physician. Athletes injured or unable to participate in regularly schedule conditioning or competitions were also excluded.

### Study Design.

A between groups parallel design study was employed to compare the effect of a novel formulation containing DHA/EPA to marine algae DHA on plasma fatty acids DHA, EPA, and ARA over the course of a season to include pre-season camp and the competitive season. American football athletes were assigned to ingest either a novel formulation of DHA/EPA (n=6) derived from Tuna Fish Oil or marine algae DHA (n=6) derived from Schizochytrium sp. containing 35-45% DHA by weight. Prior to the start of the season, blood was sampled at specific intervals coincident with changes in intensity, hours of contact, and coincident with those times in which the number and magnitude of head impacts have been reported to change. A baseline blood sample was taken when volunteers returned from a period of no contact (Baseline; T1) prior to pre-season training camp (Baseline; T1). A second sample and remaining blood samples were taken throughout the competitive season on the Monday following a Saturday game (48 hours) (T6).

Fatty Acid Analysis. Serum fatty acid concentrations of DHA, EPA, and ARA were measured by a modified method of Ostermann et al. (2014) using gas chromatography/mass spectrometry (GC/MS).

### Results

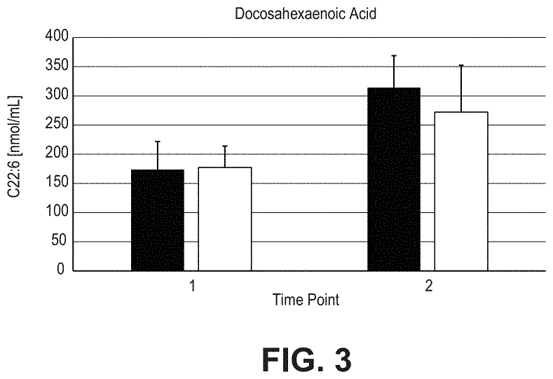

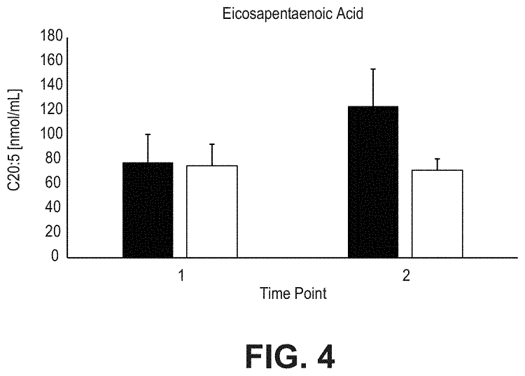

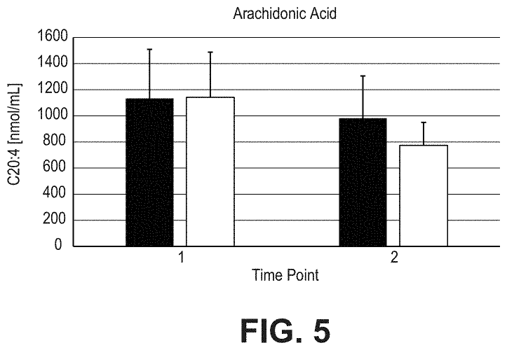

FIG. 3 shows effect of certain disclosed formulations (DHA/EPA; n=6) and algae DHA (Algae DHA; n=6) on the relative change from 1 (T1) in serum fatty acid DHA in American football athletes over the course of the season compared to the end of the season 2 (T6) [nmol/ml]. FIG. 4 shows the effect of certain disclosed formulations (DHA/EPA; n=6) and algae DHA (Algae DHA; n=6) on the relative change from 1 (T1) in serum fatty acid EPA in American football athletes over the course of the season compared to the end of the season 2 (T6) [nmol/ml]. FIG. 5 shows the effect certain disclosed formulations (DHA/EPA; n=6) and algal DHA (Algal DHA; n=6) on the relative change from 1 (T1) in serum Arachidonic acid (ARA) in American football athletes over the course of the season compared to the end of the season 2 (T6) [nmol/ml]. Relative to the marine algae DHA, the novel formulation caused an increase of serum fatty acids DHA, EPA as well as decrease of ARA, an inflammatory biomarker, showing an overall improved fatty acid absorption profile of DHA and EPA, therefore, an improved efficacy (Table 1, FIGS. 3-5).

TABLE 1
Serum fatty acid changes in a novel formulation (DHA/EPA; n = 6) and algae docosahexaenoic acid
(Algae DHA; n = 6) on the relative change from T1 in serum fatty acids DHA, EPA, and ARA in
American football athletes over the course of the season compared to the end of the season T6 [nmol/ml].

| | Arachidonic Acid | | | | | | Eicosapentaenoic Acid | | | | | | Docosahexaenoic Acid | | | | |
|---|---|---|---|---|---|---|---|---|---|---|---|---|---|---|---|---|---|
| | (C20:4)[nmol/mL] | | | | | | (C20:5) [nmol/mL] | | | | | | (C22:6) [nmol/mL] | | | | |
| | Pre | | Post | | p- | | Pre | | Post | | p- | | Pre | | Post | | p- |
| | Mean | SD | Mean | SD | value | | Mean | SD | Mean | SD | value | | Mean | SD | Mean | SD | value |
| DHA/EPA | 1133.2 | 375.1 | 981.9 | 322.9 | 0.309 | DHA/EPA | 78.0 | 22.7 | 123.9 | 30.1 | 0.043 | DHA/EPA | 173.8 | 48.1 | 314.3 | 54.7 | 0.011 |
| Algae | 1142.0 | 345.9 | 774.1 | 175.6 | 0.056 | Algae | 75.1 | 17.6 | 71.5 | 9.3 | 0.722 | Algae | 177.4 | 36.5 | 272.3 | 80.2 | 0.064 |
| DHA | | | | | | DHA | | | | | | DHA | | | | | |

### Example 4

### Comparison DHA/EPA/n-3 DPA Formulation Vs Non-Treated Control

### Methods

Subjects. All athlete volunteers were NCAA Division I American football athletes cleared to participate in university athletics as determined by the team's medical staff. Athletes using long-term antiinflammation therapy, using anti-hypertensive medications, using medications known to affect blood lipids, consuming fish oil or omega-3 fatty acid supplements, or consuming more than two servings of fish per week were excluded. Athletes under the age of 18 were also excluded. Athletes who were injured, became ill, or were unable to participate in regularly scheduled conditioning, practice, or competitions were excluded. The athletes receiving treatment, the treatment team, were members of an NCAA Division I American football team, while the athletes receiving no treatment were members of an NCAA Division III American football team. Serum NFL was compared across starters and non-starters. For purposes of the study, starters were defined as athletes known to go out with the first or second team, first or second on the depth roster, and take a majority of the repetitions. There were nineteen starters on the control team (n=19) and ten on the treatment team (n=10).

### Study Design.

A between groups parallel design study was employed to compare the effect of a novel formulation containing DHA/EPA/n-3 DPA to a non-treated control on proportion EPA, DHA, and omega-6:omega-3 fatty acid ration, and on serum NFL over the course of a season to include pre-season camp and the competitive season.

American football athletes on the treatment team were assigned to ingest a daily supplement of omega-3 fatty acids containing 2000 mg DHA, 566 mg EPA, and 330 mg n-3 DPA each day of their 2016 football season for a total of 89 days. The non-treated control team received no treatment over the same period. Both teams limited servings of foods high in omega-3 fatty acids to no more than two per week during the study.

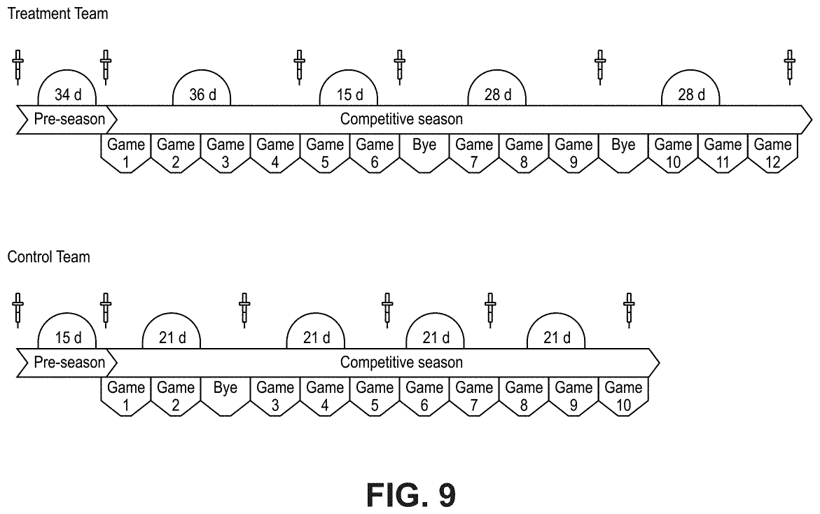

A schematic representation of the study protocol is shown in FIG. 9. Prior to the start of the season, blood was sampled at specific intervals coincident with changes in intensity, hours of contact, and coincident with those times in which the number and magnitude of head impacts have been reported to change. A baseline blood sample was taken when volunteers returned from a period of no contact, prior to initiation of supplementation and prior to pre-season training camp (Baseline; T1). A second sample was taken at the conclusion of a two-a-day preseason camp (Post-Camp; T2). Remaining blood samples were taken throughout the competitive season on the Monday following a bye week or a Saturday game (48 hours) (T3-T6).

Serum NFL Quantification. Serum NFL concentrations were measured using digital array technology on a Single Molecule Array HD-1 Analyzer, software version 1.5. The same lot of kits were used for each assay. Prior to analyses, samples were diluted ¼. All samples were above the lower limit of quantification. The lower limit of detection was 0.048 pg per ml. Duplicates were run with a median dose coefficient of variation of 5 percent.

Fatty Acid Analysis. Plasma samples were aliquoted, vortexed, and separated into phases. The lower phase was extracted, dried under nitrogen gas, and methylated. Methylated samples were then separated, and the top layer was extracted and dried under nitrogen gas. The samples were then reconstituted in hexane for fatty acid analysis. Plasma fatty acid concentrations were measured using gas chromatography, in which fatty acid peaks were identified by comparing their respective retention times to authentic fatty acid standards.

### Results

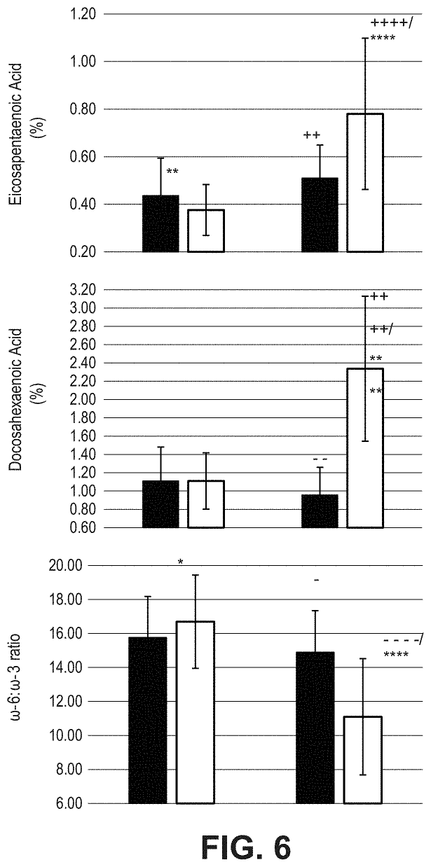

ω-3 supplementation increases serum ω-3 fatty acids and lowers ω-6: ω-3 ratio. Baseline (T1) and post-season (T6) proportion DHA, EPA, and the ω-6:ω-3 ratio is presented in FIG. 6, panels A-C, respectively. Prior to the start of camp (Baseline, Pre-Camp) and ω-3 FA supplementation, a small (ES=0.42) likely difference was observed in proportion EPA between groups, with those in the control group presenting with higher baseline (mean difference; ±90% CI; 0.06%; ±0.06%) EPA. The higher EPA observed in the control group at baseline resulted in a small (ES=0.37) possible difference (−0.93, ±1.1) in the ω-6:ω-3 ratio. At the conclusion of the season (T6) those in the treatment group had a most likely very large (ES=3.8) and most likely extremely (ES=4.8) large increases in EPA and DHA, respectively. Those increases resulted in large (ES=1.24) and very large (ES=2.62) differences between groups in EPA (0.27%; ±0.13%) and DHA (1.4%; ±0.67%), respectively, by the end of the season. The increase in proportion EPA and DHA observed in the treatment group resulted in a most likely large (ES=2.0) decrease in the ω-6:ω-3 ratio. Though a small (ES=0.47) likely increase in proportion EPA was observed in the control group over the course of the season, resulting in a small (ES=0.36) decrease in ω-6:ω-3 ratio, the larger magnitude changes observed in the treatment group resulted in a most likely large (ES=1.35) difference in ω-6:ω-3 ratio (3.8, ±1.8) between groups post-season (T6).

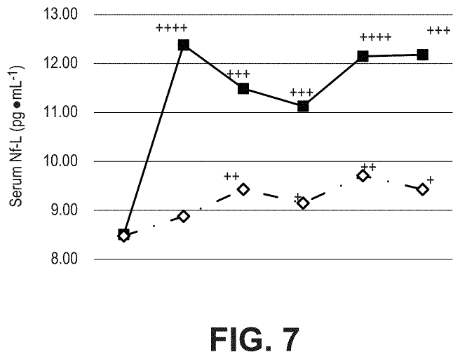

ω-3 supplementation attenuates serum neurofilament light, a surrogate marker of head trauma. FIG. 7 shows the effect of ω-3 FA supplementation on serum Nf-L. Corresponding percent change from baseline within each team with corresponding statistical estimates of fold change and qualitative inference are presented in Table 2. Differences between the control and treatment teams in serum Nf-L prior to the start of camp and ω-3 FA supplementation were unclear (0.3 pg·mL-1; ±1.4 pg·mL-1). At the conclusion of camp, a most likely increase (3.7 pg·mL-1; ±1.7 pg·mL-1) moderate in magnitude (ES=1.07), but representing a 1.5 fold increase over baseline values was observed in the control team. In contrast, only a trivial (ES=0.14) unclear increase (0.1 pg·mL-1; ±2.1 pg·mL-1) was observed receiving treatment, which resulted in a very likely difference in absolute difference (3.7 pg·mL-1; ±1.7 pg·mL-1; ES=0.71), but a most likely difference (ES=1.04) in fold change difference between the control and treatment teams. Thereafter, serum Nf-L remained elevated in those athletes not receiving ω-3 FA supplementation (ES range=0.73-1.02), control team, throughout the competitive season. Though an increase was observed in serum Nf-L in the treatment team, those increases were only small (ES range=0.23-0.43) in magnitude. The difference in magnitude of increase over the course of the season between the control and treatment team resulted in likely to mostly likely differences between teams in fold change over the course of the competitive season (Table 2).

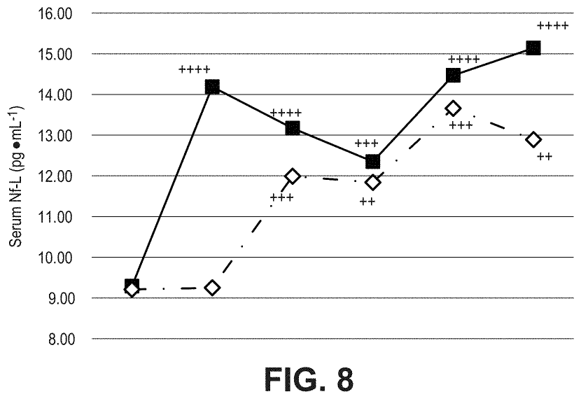

ω-3 supplementation attenuates serum neurofilament light, a surrogate marker of head trauma, in those athletes categorized as starters. The effect of ω-3 FA supplementation on serum Nf-L in those American football athletes categorized as starters is presented in FIG. 8. Corresponding percent change from baseline within each team's starters and corresponding statistical estimates of fold change and qualitative inference are presented in Table 2. Similar to that observed in all athletes, differences between the starters on the control and treatment teams in serum Nf-L prior to the start of camp and ω-3 FA supplementation were unclear (0.08 pg·mL-1; ±2.2 pg·mL-1). Further similar, at the conclusion of camp, a most likely increase (4.9 pg·mL-1; ±2.2 pg·mL-1; ES=1.10) of moderate magnitude was observed in serum Nf-L for those starters on the control team, which represented a larger fold change (1.7) over baseline values than when non-starters were included. An increase was not observed in those starters receiving ω-3 FA supplementation (0.04 pg·mL-1; ±2.1 pg·mL-1; ES=0.02) post-camp (T2). That difference resulted in a most likely difference (4.9 pg·mL-1; ±3.0 pg·mL-1; ES=0.96). Thereafter, serum Nf-L remained elevated in those starters on the control team (ES range=0.69-1.32) throughout the competitive season. When non-starters were excluded, larger mean increases were observed in those receiving ω-3 FA supplementation (ES range=1.12-1.89); however, those increases were again of lower magnitude compared to the control team starters (FIG. 8; Table 3).

### Figure Legends

FIG. 6 shows the effect of certain disclosed formulations (DHA/EPA/n-3 DPA; n=31) on the proportion EPA, DHA, and omega-6:omega-3 ratio. Threshold for smallest substantial change calculated as 0.2 times baseline SD_{between}; Likelihood is shown as increased number of symbols (+ used for example) and represented by unique symbol: (+) relative to baseline, (*) relative to control. + possible, ++ likely, +++ very likely, ++++ most likely. All data are mean±SD. FIG. 7 shows the effect of certain disclosed formulations (DHA/EPA/n-3 DPA; n=31) on serum NFL (pg·mL-1). Threshold for smallest substantial change calculated as 0.2 times baseline SD_{between}; Likelihood is shown as increased number of symbols: (+) relative to baseline. + possible, ++ likely, +++ very likely, ++++ most likely. All data are mean. Corresponding percent change from baseline within each team with corresponding statistical estimates of fold change and qualitative inference are presented in Table 2. Supplementation with the DHA/EPA/n-3 DPA formulation increased serum omega-3 fatty acids and lowered the omega-6:omega-3 ratio (FIG. 6). FIG. 7 shows the effect of certain disclosed formulations (DHA/EPA/n-3 DPA; n=31) on serum NFL (pg·mL-1). Threshold for smallest substantial change calculated as 0.2 times baseline SD_{between}; Likelihood is shown as increased number of symbols: (+) relative to baseline. + possible, ++ likely, +++ very likely, ++++ most likely. All data are mean. Corresponding percent change from baseline within each team with corresponding statistical estimates of fold change and qualitative inference are presented in Table 2. Supplementation with the above formulation also attenuated serum NFL (Table 2, FIG. 7). FIG. 8 shows the effect of certain disclosed formulations (DHA/EPA/n-3 DPA; n=31) on serum NFL (pg·mL-1) in those American football athletes categorized as starters. Threshold for smallest substantial change calculated as 0.2 times baseline SD_{between}; Likelihood is shown as increased number of symbols: (+) relative to baseline. + possible, ++ likely, +++ very likely, ++++ most likely. All data are mean. Corresponding percent change from baseline within each team with corresponding statistical estimates of fold change and qualitative inference are presented in Table 3. Supplementation further attenuated serum NFL in athletes categorized as starters. (Table 3, FIG. 8).

TABLE 2
Percent change (%) from baseline in serum NFL over the course of the season in American
football athletes with statistical estimates and qualitative inference.

| | Post-C [symbol] mp (T2) | Competition (T3) | Competition (T4) | Competition (T5) | Competition (T6) |
|---|---|---|---|---|---|
| Control [symbol] | 51.5 ± 48.5 | 41.7 ± 43.0 | 39.3 ± 46.4 | 50.1 ± 85.0 | 47.2 ± 87.1 |
| Within Fold Change [symbol] | 1.5; x/+1.2 | 1.4; x/+1.2 | 1.4; x/+1.2 | 1.5; x/+1.2 | 1.4; x/+1.2 |
| Inference [symbol] | Most Likely | Very Likely | Very Likely | Most Likely | Very Likely |
| Treatment [symbol] | 4.7 ± 40.5 | 9.3 ± 35.5 | 6.3 ± 41.7 | 12.3 ± 68.3 | 11.0 ± 60.8 |
| Within Fold Change [symbol] | 1.0; x/+1.2 | 1.1; x/+1.1 | 1.1; x/+1.1 | 1.2; x/+1.2 | 1.2; x/+1.2 |
| Inference [symbol] | Unclear | Possibly | Possibly | Likely | Possibly |
| Between Fold Change [symbol] | 1.5; x/+1.2 | 1.3; x/+1.2 | 1.3; x/+1.2 | 1.3; x/+1.3 | 1.2; x/+1.3 |
| Inference [symbol] | Most Likely | Likely | Likely | Likely | Likely |

[symbol]

Percent change (%) from baseline in serum neurofilament light with corresponding SD. [symbol]
Within-subjects fold change from baseline. [symbol]
Between-groups fold change from baseline. [symbol]
90% CL; Multiply or divide as a factor of the mean to obtain the upper and lower confidence limits. [symbol]
Magnitude-based inference about the true value for outcomes where the threshold for smallest substantial change was calculated as 0.2 times baseline between-subjects SD. [symbol]
indicates data missing or illegible when filed

TABLE 3
Percent change (%) from baseline in serum NFL over the course of the season in American football
athletes categorized as starters with statistical estimates and qualitative inference.

| | Post-C [symbol] mp (T2) | Competition (T3) | Competition (T4) | Competition (T5) | Competition (T6) |
|---|---|---|---|---|---|
| Control [symbol] | 64.7 ± 55.9 | 54.9 ± 49.3 | 47.5 ± 55.7 | 70.6 ± 106.0 | 73.9 ± 106.1 |
| Within Fold Change [symbol] | 17; x/+1.2 | 1.6; x/+1.2 | 1.5; x/+1.2 | 1.7; x/+1.3 | 1.7; x/+1.3 |
| Inference [symbol] | Most Likely | Most Likely | Most Likely | Most Likely | Very Likely |
| Treatment [symbol] | 0.2 ± 19.8 | 26.1 ± [symbol] .47 | 23.75 ± 63.13 | 42.9 ± 115.0 | [symbol] 6.6 ± 95. [symbol] |
| Within Fold Change [symbol] | 1.0; x/+1.0 | 1.3: x/+1.2 | 1.2; x/+1.2 | 1.4; x/+1.4 | 1.4; x/+1.4 |
| Inference [symbol] | Unclear | Likely | Likely | Likely | Likely |
| Between Fold Change [symbol] | 1.6; x/+1.3 | 1.3; x/+1.2 | 1.2; x/+1.3 | 1.3; x/+1.6 | 1.4; x/+1.5 |
| Inference [symbol] | Most Likely | Likely | Likely | Unclear | Likely |

[symbol]

Percent change (%) from baseline in serum neurofilament light with corresponding SD. [symbol]
Within-subjects fold change from baseline. [symbol]
Between-groups fold change from baseline. [symbol]
90% CL; Multiply or divide as a factor of the mean to obtain the upper and lower confidence limits. [symbol]
Magnitude-based inference about the true value for outcomes where the threshold for smallest substantial change was calculated as 0.2 times baseline between-subjects SD. [symbol]
indicates data missing or illegible when filed

### Example 5

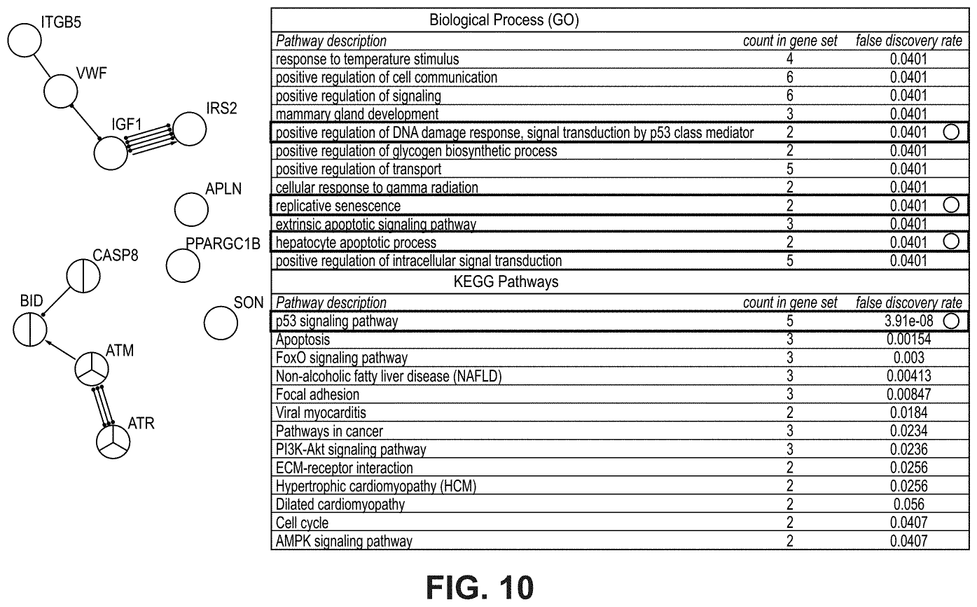

To explore the underpinnings of the synergistic effects achieved by the presently disclosed composition, relative to the effects of DHA alone, we employed high-volume data extraction methodology for identifying protein interaction networks for both compositions and for determining network overlaps between them. Specifically, the effects of DHA and DHA (2,000 mg), EPA (560 mg), and 320 mg docosapentaenoic acid, 20 mg of other omega-3 fatty acids (hereinafter FA-101) were compared. FIG. 10 shows the area of the protein interaction networks that does not overlap and thus identifies differences between these two products. Thus, the protein network fragment shown on the left side of FIG. 10 identifies circuits that are preferentially modulated by FA-101 and the right side of FIG. 10 identifies molecular processes that are directly affected by these protein network fragments. To assess the impact of this protein interaction network difference on the functions of >3400 circuits regulating the body's response to chronic brain injury we examined protein interaction network overlaps between molecular processes shown in FIG. 10 and the 3400 molecular processes affected by traumatic brain injury. For identifying specific circuits affected by the FA-101 and not the DHA formula we examined the crosslinking pattern of >3400 molecular processes. The results indicated that the regulation of neuronal stem cell differentiation, which plays a key role in in the pathogenesis of ischemia seems to be differentially affected by the two products. In this regard the regulation of neuronal stem cell proliferation is regulated by mitochondrial functions. Thus, EPA, but not DHA, significantly increased proliferation of Neuronal stem cells compared to controls, an effect associated with enhanced levels of the endocannabinoid 2-arachidonylglycerol (2-AG) and p-p38 MAPK. EPA, a component of the FA-101 formula, safeguards mitochondrial membrane potentials and protects mitochondria against oxidative and inflammatory stress. In contrast, DHA activates mitochondrial biogenesis, increases oxidative stress and dissipate mitochondrial membrane potential. For counteracting this effect which compromises neuronal integrity, DHA, upon PLA2 mediated release form cell membranes, is rapidly converted to DHA metabolites “Neuroprotectin D1” and “4-hydroxyhexenal”. Both of these DHA metabolites have EPA like functions and protect cells from oxidative stress and counteract pro inflammatory signals.

Accordingly, since EPA, and the DHA metabolites exert similar neuroprotective protective functions, the interaction between EPA and DHA metabolites may manifest itself as synergy which means that the FA-101 formula will have superior therapeutic properties compared with DHA. Adding further advantage of FA-101 are effects on protein network circuits showing that FA-101 differentially regulates metabolism. For example, in cultured rat hepatocytes, the oxidation of [1-14C]palmitic acid is reduced by DHA, whereas it is stimulated by EPA.

The significance of this functionality for the treatment of traumatic brain injury is that there is an increased reliance on glycolysis during ischemia and fatty acid β-oxidation during reperfusion following ischemia as sources of adenosine triphosphate (ATP) production. FA-101, but not DHA, is shown to increase mitochondrial fatty acid oxidation and to upregulate the enzyme 2,4-dienoyl-CoA reductase gene which plays a key role in these metabolic processes. Apoptotic cell death, proposed to play a role in the neuronal loss observed following traumatic injury in the CNS, is induced by palmitic and stearic acid. EPA but not DHA is able to lower the concentration of these fatty acids and hence exert neuroprotective effects.

Lastly, expanding the portfolio of advantageous FA-101 properties is the observation that the FA-101 formula has the capacity to affect microglia differentiation. Microglial cells, trough activation of cPLA2 release DHA from the cell membrane and trigger pro inflammatory responses. Increased inflammation and reduced neurogenesis have been associated with depression which is one of the main comorbidities of chronic traumatic brain injury. In addition, microglia activation and concomitant release of the proinflammatory cytokine Interleukin-1 (3 exacerbate neuro-degeneration and to spatial and contextual memory.

Although the disclosure has been described with reference to preferred embodiments, persons skilled in the art will recognize that changes may be made in form and detail without departing from the spirit and scope of the disclosed apparatus, systems and methods.

What is claimed is:

1. A method of attenuating trauma-related brain injury in a subject comprising administering to the subject a composition comprising:
a. docosahexaenoic acid (DHA); and
b. eicosapentaenoic acid (EPA), wherein the DHA and EPA are present in a ratio ranging from about 5:1 to about 1:1.

2. The method of claim 1, wherein the composition is administered in a therapeutically effective amount.

3. The method of claim 1, wherein the composition is administered in a prophylactically effective amount.

5. The method of claim 4 wherein the DHA and EPA are present in a ratio of about 3.5:1.

6. The method of claim 5, wherein the composition further comprises docosapentaenoic acid (DPA), wherein DHA and DPA are present in a ratio of about 7:1.

7. The method of claim 5 wherein the composition comprises an emulsion.

8. The method of claim 1 wherein the composition is administered in a dose of about 2 g per day.

9. The method of claim 1, wherein the composition comprises from about 750 mg to about 6,000 mg DHA.

10. The method of claim 1 wherein, the composition is administered to a subject at high risk for sub-concussive injury.

11. The method of claim 10, wherein the composition is administered to a subject at high risk for concussive injury.

12. The method of claim 11, wherein the composition is administered prior to the high risk of concussive injury and at regular intervals following the onset of risk for concussive injury.

13. The method of claim 1, wherein administration of the composition to the subject
results in a synergistic reduction in a concussive biomarker relative to subjects
administered DHA or EPA alone, wherein the concussive biomarker is serum Neurofilament Light (NFL).

14. The method of claim 1, wherein administration of the composition to the subject results in a synergistic increase in plasma levels of DHA and/or EPA relative to subjects administered comparable amounts of DHA or EPA alone.

15. The method of claim 1, wherein the composition is administered a nutritional supplement.

16. The method of claim 1, wherein the composition is administered a functional food.

17. A method for attenuating the impact of repeated head impacts on the brain of a subject in need thereof comprising administering to the subject a composition comprising:
a. docosahexaenoic acid (DHA);
b. eicosapentaenoic acid (EPA); and
c. docosapentaenoic acid (DPA)
wherein the ratio of DHA:EPA:DPA is about 7:2:1.
